(19) United States
(12) Patent Application Publication
(10) Pub. No.: US 2020/0338289 A1
(43) Pub. Date: Oct. 29, 2020

(54) SYSTEM FOR SUPPLYING RESPIRATORY GAS AND METHOD

(71) Applicant: Loewenstein Medical Technology S.A., Luxembourg (LU)

(72) Inventors: Matthias SCHWAIBOLD, Karlsruhe (DE); Bjoern TIEMANN, Hamburg (DE); Christof GOEBEL, Hamburg (DE); Benno DOEMER, Ettlingen (DE); Benjamin ADAMETZ, Hamburg (DE)

(21) Appl. No.: 16/853,323

(22) Filed: Apr. 20, 2020

(30) Foreign Application Priority Data
Apr. 24, 2019 (DE) 102019110631.1

(51) Int. Cl. A61M 16/00; A61M 16/16; A61M 16/06

(52) U.S. Cl. CPC A61M 16/022; A61M 16/0057; A61M 16/16; A61M 2205/3368; A61M 16/06; A61M 2016/0027; A61M 2202/0208; A61M 16/0666

(57) ABSTRACT

A system and method for supplying respiratory gas comprises a ventilation device comprising a respiratory gas source and a control device configured for generating a defined respiratory gas flow for ventilation by the ventilation device and for monitoring a parameter characteristic of a pressure by a sensor device. The parameter characterizes that pressure at which the ventilation device admits the respiratory gas stream for maintaining the defined respiratory gas flow in order to counterbalance breathing activity of the patient. The control device is configured for registering a course of the parameter over time as a pressure profile.

### CROSS-REFERENCE TO RELATED APPLICATIONS

The present application claims priority under 35 U.S.C. § 119 of German Patent Application No. 102019110631.1, filed Apr. 24, 2019, the entire disclosure of which is expressly incorporated by reference herein.

### BACKGROUND OF THE INVENTION

### 1. Field of the Invention

The present invention relates to a system for supplying respiratory gas and to a method for operating such a system. The system comprises at least one ventilation device having at least one respiratory gas source for generating a respiratory gas stream and at least one control device. The control device generates a defined respiratory gas flow for flow-controlled ventilation by means of the ventilation device.

### 2. Discussion of Background Information

In the case of volume-controlled ventilation, a defined respiratory volume is administered to the patient within a specified inspiration time. The respiratory volume is, for example, calculated from tidal volume and respiratory rate. In this form of ventilation, the instrument used thus essentially attempts to administer the volume that is set.

In contrast, in the case of pressure-supported ventilation, the patient's drive to breath is taken into account. When the patient inhales, the breathing process is supported and the work of breathing is thereby facilitated. In pressure-supported ventilation, a lower pressure level and an upper pressure level are generally determined. In most cases, these two pressures are variably settable. The tidal volume then arises from the quantity of air that has been delivered until the upper pressure is reached.

However, it has become apparent that control or regulation in the case of instruments for pressure-controlled or volume-controlled ventilation is highly complicated or in great need of improvement. In pressure-controlled or volume-controlled ventilation, the system causes the proportion of pendelluft to be relatively large, pendelluft being that air which is moved to and fro as a result of ventilation (and breathing) and does not participate in gas exchange.

By contrast, the flow-controlled ventilation according to the invention flushes out the upper respiratory tract at least in part and thus reduces the proportion of pendelluft.

In view of the foregoing, it would be advantageous to be able to make flow-controlled ventilation possible. Preferably, it would be advantageous if an improved controllability of the ventilators and also a more reliable monitoring of the breathing activity of the patient were made possible. In particular, it would be advantageous if improved ways of preventing the mucous membranes from drying out were provided.

### SUMMARY OF THE INVENTION

The present provides a system and a method as set forth in the independent claims. Further developments and advantageous embodiments are subject matter of the dependent claims. Further advantages and features will be revealed by the general description and the description of the exemplary embodiments.

The system according to the invention serves for supplying respiratory gas or ventilation of a patient. The system comprises at least one ventilation device having at least one respiratory gas source for generating a respiratory gas stream. The system further comprises at least one control device which is suitable and designed for generating at least one defined respiratory gas flow for ventilation by means of the ventilation device and for preferably regulating said respiratory gas flow to a desired value and especially to a certain flow rate. In this connection, the control device is suitable and designed for monitoring at least one parameter characteristic of a pressure by means of at least one sensor device. The parameter characterizes that pressure at which the ventilation device admits the respiratory gas stream for maintaining the defined respiratory gas flow in order to counterbalance and especially compensate for breathing activity of the patient. In this connection, the control device is especially suitable and designed for registering a course of the parameter over time as a pressure profile.

The system according to the invention offers many advantages. A considerable advantage is offered by the monitoring and registration of the parameter and that what is monitored to this end is that pressure which must be set by the ventilation device to counterbalance the breathing activity. This makes it possible for the ventilation and also the breathing activity of the patient to be monitored in a particularly uncomplicated manner and, at the same time, in a very reliable manner. Moreover, this allows a particularly reliable and, at the same time, constructively uncomplicated way of regulating the ventilation device depending on the breathing activity of the patient.

The control device is preferably suitable and designed for determining at least one characteristic variable that is characteristic of the breathing activity of the patient from the pressure profile (taking into account the pressure profile). Such a characteristic variable can, for example, be tidal volume, breath duration, respiratory rate or the like. The characteristic variable can also be an apnoea-hypopnoea index (AHI). Particularly preferably, the characteristic variable is a breathing phase, and so preferably an exhalation and inhalation are identified and distinguished from one another. This offers a particularly reliable monitoring of the ventilation. Moreover, such a characteristic variable can be used particularly advantageously for regulating the ventilation.

Preferably, the control device is suitable and designed for identifying an inhalation (the breathing phase of inhalation) by the fact that the pressure required for maintaining the defined respiratory gas flow decreases over time and especially decreases by a defined extent and/or changes by a defined extent. In particular, the pressure profile shows a negative slope for identifying the inhalation. It is possible that derivatives of the pressure profile are evaluated to this end and/or other analysis techniques for functions are used. It is also possible that the pressure required for maintaining the defined respiratory gas flow must drop and/or must fall below a defined threshold so that an inhalation is identified. In particular, to this end, the pressure drops by a certain extent compared to at least one previously registered value and/or a previously registered pressure profile. It is possible that the pressure profile must have a defined pressure difference in order to identify the inhalation.

It is also preferred that the control device is suitable and designed for identifying an exhalation (the breathing phase of exhalation) by the fact that the pressure required for maintaining the defined respiratory gas flow increases over time and especially increases by a defined extent and/or changes by a defined extent. In particular, the pressure profile shows a positive slope for identifying the exhalation.

It is possible that derivatives of the pressure profile are evaluated to this end and/or other analysis techniques for functions are used. It is also possible that the pressure required for maintaining the defined respiratory gas flow must rise and/or must rise above a defined threshold so that an exhalation is identified. In particular, to this end, the pressure rises by a certain extent compared to at least one previously registered value and/or a previously registered pressure profile. It is possible that the pressure profile must have a defined pressure difference in order to identify the exhalation.

Such embodiments offer a way of identifying the breathing phase, or an inhalation or exhalation, that is particularly reliable and, at the same time, uncomplicated in a sensor-based manner. Moreover, the identification of inhalation or exhalation is particularly crucial for a specific regulation of the ventilation device taking into account the breathing situation of the patient.

In particular, what is effected depending on the identified breathing phase is measurement of the respiratory rate and/or controlling of the admixing of oxygen (especially inspiratory more than expiratory) and/or controlling of the humidification and heating of respiratory gas (especially inspiratory more than expiratory) and/or controlling of the respiratory gas flow (especially inspiratory more than expiratory (bilevel high-flow)). For the administration of the bilevel high-flow, controlling is effected such that a higher flow rate is specified during inspiration in order to increase the tidal volume, for example within the range of 40-60 l/min. Analogously, controlling is effected with a lower flow rate during expiration in order to only just ensure a flush-out of the dead space of the upper respiratory tract, preferably within the range of 10-40 or 5-38 l/min.

According to the invention, the control device is also suitable and designed for identifying a disconnection (detachment of the interface from the nose or of the interface from the hose or of the hose from the instrument). The control device identifies a disconnection either through the occurrence of a long apnea, which, for example, lasts longer than one minute or preferably longer than two minutes, or/and through a lowering of the required rotational speed/pressure for achieving the flow rate that is set. In the event of identification of a disconnection, the control device is suitable and designed for triggering a disconnection alarm and/or switching off the admixing of oxygen and/or reducing the flow rate.

According to the invention, what is also provided is administering the high-flow mode via a normal respiratory mask instead of via a nasal cannula. In this specific application, the fan device counteracts the breathing activity of the patient—it attempts to generate a constant flow. This can be used as a training program for the respiratory muscles. The higher the flow that is set, the greater the resistance (training) felt by the patient when exhaling. The lower the flow that is set, the greater the resistance (training) felt by the patient when inhaling. According to the invention, the total severity of the training can be set by the control system (via the regulator coefficients of the high-flow regulators). The more passive/cautious the regulation, the lower the resistance when breathing (inhalation/exhalation in equal measure). The more dynamic/aggressive the regulation, the higher the resistance. Via telemonitoring, it is possible to capture the daily duration of the respiratory muscle training.

In a particularly advantageous further development, the control device is suitable and designed for determining at least one breathing event on the basis of the pressure profile. The breathing event is determined especially by comparison with stored pressure profiles. Such breathing events are, for example, snoring, flattening of breathing and/or obstructive pressure peaks, apneas, hypopneas or other relevant events.

It is possible and preferred that the control device is suitable and designed for determining at least one breathing pattern on the basis of the pressure profile. Preferably, the control device is suitable and designed for examining the breathing pattern for at least one breathing event. The breathing pattern is examined especially by comparison with at least one stored breathing pattern and/or by means of at least one (teachable) algorithm. The breathing pattern corresponds especially to a characteristic temporal course of the breathing and preferably the breathing events occurring therein. For example, by examining a breathing pattern, it is possible to make an identification of being awake or being asleep. A breathing pattern is, for example, characterized by the sequence of apneas and/or hypopneas.

It is possible that the control device is suitable and designed for checking the calculated pressure profile and/or the calculated breathing pattern and/or an identified breathing event for plausibility. To this end, it is possible to use known methods for evaluating pressure profiles or events in the context of ventilations. For example, a specified significance level must be reached so that a certain breathing event and/or breathing pattern is identified.

In particular, the control device is suitable and designed for controlling the ventilation device on the basis of the pressure profile, as is known, for example, from pressure-supported or pressure-controlled ventilation. Depending on the pressure profile, especially at least one of the following settings or regulations is performable: auto-start, auto-stop, ramp, identification of being awake, event identification, statistics, rate, respiratory volume, apneas, hypopneas, event control, saturation control, automatic flow-rate control, telemonitoring, close monitoring, control or setting of PSG and/or SpO2 and/or TcCO2, soft PAP, admixing of oxygen. In particular, what is possible depending on the pressure profile is at least one mode of control known from CPAP, IPAP or BiPAP ventilation.

The control device can be suitable and designed for outputting at least one user interaction and, for example, an alarm depending on the pressure profile. It is also possible that the control device is suitable and designed for transmitting the pressure profile and especially a breathing event and/or breathing pattern identified by means thereof to at least one network device. In particular, the system is designed for telemonitoring. In particular, it is possible to prepare and/or transmit therapy statistics by means of the control device depending on the pressure profile.

In all embodiments, it is particularly preferred that the control device is suitable and designed for setting at least one instrument parameter of the ventilation device depending on the pressure profile and/or for setting at least one ventilation parameter by means of the ventilation device. In particular, the ventilation device is controllable depending on the pressure profile. In particular, the control device is suitable and designed for setting the instrument parameter and/or ventilation parameter as a response to a breathing event and/or a breathing pattern. The instrument parameter encompasses, for example, a fan rotational speed and/or a humidification output and/or a heating output or the like. The instrument parameter can also encompass a switch-on and/or switch-off and/or pause of the ventilation device. The instrument parameter can also concern software settings. The ventilation parameter is preferably a flow or a flow rate and/or a pressure and/or some other parameter characteristic of the ventilation. The instrument parameter and the ventilation parameter can be coupled and/or influence one another. It is possible that the instrument parameters are set in order to realize a certain ventilation parameter.

Preferably, the instrument parameter and/or ventilation parameter is set depending on at least one identified breathing pattern and/or at least one breathing event. In particular, the flow rate of the respiratory gas flow is set depending on the identified breathing pattern and/or breathing event.

In an advantageous embodiment, the control device is suitable and designed for setting at least one flow rate of the respiratory gas flow depending on a saturation of the respiratory gas flow with oxygen by means of the ventilation device. It is also possible that at least one other ventilation parameter is settable depending on the oxygen saturation. In particular, the oxygen saturation is sensor-monitored. In particular, the oxygen saturation is sensor-capturable. It is also possible that the flow rate of the respiratory gas flow is settable depending on an admixing of oxygen that is performed by the system and/or is permanently set.

The control device is preferably suitable and designed for setting an admixing of oxygen into the respiratory gas stream depending on the pressure profile. Preferably, the control device is suitable and designed for modulating the admixing of oxygen in a breathing phase-dependent manner and especially for modulating the admixing of oxygen in a breathing phase-dependent manner depending on the pressure profile. In particular, the admixing of oxygen is specifically increasable and/or decreasable depending on the pressure profile. In particular, at least one specifically openable or closable oxygen source is provided for the admixing of oxygen. The admixing of oxygen can be up to 100%. It is also possible that the admixing of oxygen is deactivated or is 0%. The admixing of oxygen can, for example, be up to 300 l/min.

In particular, the admixing of oxygen is effected according to at least one of the following instructions: hold the proportion of oxygen, hold the absolute flow of oxygen, demand-based control from measured or estimated patient requirements. In particular, at least one sensor-based regulation is provided for the admixing of oxygen. The sensor-based regulation encompasses especially SpO2-based regulation and/or TcCO2-based regulation and/or other modes of regulation.

Particularly preferably, the admixing of oxygen and/or an oxygen content in the respiratory gas flow is decreased by a certain extent when a flow rate of the respiratory gas flow is increased. According to clinical experience, such an inverse adjustment has been found to be particularly advantageous. Owing to the correspondingly high flow rate in flow-controlled ventilation, a better oxygen uptake is achieved, meaning that admixing can be correspondingly decreased. In particular, a rise in the flow rate of the respiratory gas flow is accompanied by a decrease in the admixing of oxygen or in the oxygen content in the respiratory gas flow. In particular, at least one assignment function describing a reversed proportionality between the admixing of oxygen and the flow rate is stored.

In particular, the admixing of oxygen only takes place during an inhalation. In particular, the admixing of oxygen does not take place during an exhalation. It is possible and preferred that the admixing of oxygen is increased during an inhalation compared to an exhalation. In this connection, the inhalation and exhalation are preferably identified on the basis of the pressure profile as described above. Such an enhanced admixing during inspiration offers many advantages. A sensor-based regulation is, however, also possible.

The applicant reserves the right to claim a system for supplying respiratory gas or ventilation of a patient. Such a system comprises at least one ventilation device having at least one respiratory gas source for generating a respiratory gas stream. The system comprises at least one control device which is suitable and designed for generating at least one defined respiratory gas flow for volume-controlled ventilation by means of the ventilation device and for preferably regulating said respiratory gas flow to a desired value and especially to a certain flow rate. In this connection, the system comprises at least one humidification device for especially specific humidification of the respiratory gas stream and/or at least one heating device for especially specific heating of the respiratory gas stream. Such a system is preferably designed as the above-described system. Preferably, the above-described system is also designed in this manner.

The humidification device comprises, for example, a nebulizer and/or moistener and/or humidifier and/or vaporizer. It is possible that the heating device is also suitable for cooling the respiratory gas stream. It is possible that the humidification device is also suitable for dehumidifying the respiratory gas stream.

In particular, the control device is suitable and designed for setting a humidification and/or heating of the respiratory gas stream depending on the pressure profile and especially for regulating it/them to a target temperature and/or target humidity of the respiratory gas flow. The target temperature and/or target humidity is measured especially on the patient and/or near the patient. For example, the target temperature and/or target humidity is measured at an outlet region of a hose connection to the patient and/or in the region of a breathing interface. A measurement at another site is also possible, for example at an instrument outlet or at an outlet of the ventilation device. In particular, at least one sensor device having appropriate sensors is provided for capturing the target temperature and/or target humidity. Humidification is effected especially with the above-described humidification device. Heating is effected especially with the above-described heating device.

Preferably, breathing phase-controlled humidification and/or heating is settable by means of the control device. In this connection, the breathing phase is preferably ascertained on the basis of the pressure profile as described above.

The control device is especially suitable and designed for setting the humidification and/or heating depending on a flow rate of the respiratory gas flow. Such an adjustment of the humidification and/or heating to the flow rate is particularly advantageous, since dry and/or cold streams are often felt as particularly uncomfortable in the case of the correspondingly high flow rates of the high-flow.

The control device is preferably suitable and designed for setting the humidification and/or heating depending on at least one sensor-captured variable. In this connection, the variable is preferably taken from a group of variables, the group comprising at least: ambient temperature, ambient humidity, temperature in the system, humidity in the system, hose temperature, temperature at a patient interface, humidity at the hose end and/or at a patient interface. The temperature or humidity in the system can, for example, be picked off in the region of the heating devices, especially at a heating rod and/or plate and/or sensor. The temperature or humidity in the system can also be picked off within the humidification device and/or in/on a housing and/or on a fan device and especially a fan outlet.

In a particularly advantageous embodiment, the control device is suitable and designed for at least approximately compensating for flow errors caused by the humidification and/or heating and/or other deviation by means of the ventilation device. In particular, flow errors between 0.1% and 10% are compensatable. In particular, flow errors of a few percent are compensatable. In particular, flow errors caused by the humidification or vaporization of water are compensatable.

In a particularly advantageous further development, the control device is suitable and designed for reducing the flow rate of the respiratory gas flow that is intended for the ventilation until a measure of the humidity of the respiratory gas flow reaches a threshold and/or until the temperature of the respiratory gas flow reaches a threshold. In this connection, the flow rate of the respiratory gas flow that is intended for the ventilation is reducible especially by a stored extent and/or to a stored value. In particular, what is then completely provided upon reaching and/or exceeding the threshold is the flow rate intended for the ventilation.

Particularly preferably, the flow rate of the respiratory gas flow that is intended for the ventilation is only provided when a measure of the humidity of the respiratory gas flow and/or the temperature of the respiratory gas flow reaches a threshold. Preferably, a humidity-controlled ramp is stored for the flow rate of the respiratory gas flow.

In a particularly advantageous further development, the system comprises a humidification device for humidification of the respiratory gas stream and at least one heating device for heating of the respiratory gas stream, the heating device being suitable and designed for heating the respiratory gas stream (at least during inspiration) to 36-38° C. and the humidification device being suitable and designed for humidifying the respiratory gas stream with a relative humidity within the range of 90-100%, preferably 95-99%.

Such embodiments offer particularly comfortable ventilation, since the humidification and heating have a considerable influence on the comfort of ventilation. Particularly advantageously, the flow rate intended for the ventilation is set only when humidification is ensured, since high-flow ventilation may possibly be painful without sufficient humidification. It is advantageous to heat at a relatively low flow rate and a highest possible humidity or 100% humidity at first and to increase the flow rate only afterwards.

In an advantageous embodiment, at least one drying mode is performable in the follow-up to ventilation. In particular, what is effected in this connection is drying of a hose device and especially of a ventilation hose. In particular, what is specifically removed in this connection is condensation water. The drying mode can be performable by means of the heating device and/or humidification device.

It is possible that at least one condensation protection is provided. To this end, 95% relative humidity is held at 37° C. for example. Other combinations of humidity and temperature are also possible.

The humidification device is especially suitable and designed for withdrawing the water intended for the humidification from at least one reservoir prior to heating and for heating it separately from the reservoir. In this connection, water is to be especially understood as a synonym for other liquids suitable for humidification. The separation of the water can, for example, be effected in the principle of a bird bath. Capillary effects in particular are used for separation. It is possible that the heating device comprises at least one heating plate which comprises at least one heating element and for example a heating plate, which heating element is arranged in a float with a small basin. The water intended for the humidification can also be nebulized. To this end, a mesh nebulizer and/or nozzle nebulizer and/or rotation nebulizer and/or a nebulizer with heated hose is provided for example. At the same time, an appropriate regulation system or safeguard ensuring that no mist gets into the patient is preferably designed.

The applicant reserves the right to claim a system for supplying respiratory gas or ventilation of a patient. Such a system comprises at least one ventilation device having at least one respiratory gas source for generating a respiratory gas stream. The system comprises at least one control device which is suitable and designed for generating at least one defined respiratory gas flow for volume-controlled ventilation by means of the ventilation device and for preferably regulating said respiratory gas flow to a desired value and especially to a certain flow rate. In this connection, the system comprises at least one hose device having at least one ventilation hose couplable to the ventilation device, which ventilation hose makes it possible to supply the respiratory gas stream to a patient interface. Such a system is preferably designed as one of the above-described systems. Preferably, the above-described systems are also designed in this manner.

The hose device can comprise at least one heatable ventilation hose. In particular, the heatable ventilation hose comprises at least one part of a sensor unit for capturing at least one variable characteristic of the humidity and/or temperature. In particular, the part of the sensor unit is accommodated within the ventilation hose and/or integrated therein. Said part of the sensor unit can also be arranged outside the ventilation hose at least in part or even completely. In this case, said part of the sensor unit is preferably arranged externally on the ventilation hose. The variable capturable by means of the sensor unit is usable especially for controlling or regulating the heating of the hose. In particular, the variable captured in this manner is used to regulate the heating of the hose. It is possible that at least one further variable is used for heating the hose.

Preferably, the hose device comprises at least one ventilation hose which is gradually heatable over its length. In particular, a heating gradient or temperature gradient is settable over the hose length. In particular, the ventilation hose has at least one more strongly heatable first segment which is suitable and designed for vaporizing water. In particular, the ventilation hose comprises at least one second segment which is heated to a lesser extent compared to the first segment, meaning that a specific compensation of heat losses in relation to the vaporized water is achieved in the second segment.

In a particularly advantageous embodiment, the hose device comprises at least one heatable ventilation hose having at least one heater separable from the ventilation hose. In this connection, the heater is equippable and further usable as intended with a new ventilation hose in the event of a hose exchange. This offers a particularly economical and sustainable use of a heatable ventilation hose. It is also possible that the sensor unit of the ventilation hose is separable therefrom and further usable as intended after a hose exchange.

The hose device preferably comprises at least one ventilation hose composed of a water-channeling material. For example, the material is designed analogously to a climate-control film in house construction and/or to a climate-control textile in functional clothing.

The hose device can comprise at least one heat-insulated ventilation hose. In particular, the ventilation hose is double-walled. In particular, air pockets and/or vacuumed regions are provided between at least two walls. It is also possible that the ventilation hose is surrounded by a heat-insulating material. In particular, heat insulation with respect to the environment is provided.

In all embodiments of the system, it is particularly preferred that the control device is suitable and designed for administering at least one defined respiratory volume within a specified inspiration time by means of the ventilation device through the defined respiratory gas flow. In particular, the respiratory minute volume is, in this connection, calculated from the tidal volume and the respiratory rate.

The control device is preferably suitable and designed for taking into account at least one stored maximum pressure for the defined respiratory gas flow. The maximum pressure can be fixed and/or be dynamically adjustable. In particular, the maximum pressure is dynamically adjusted depending on the pressure profile. It is also possible that the maximum pressure is manually settable. It is possible that exceeding of the maximum pressure is only possible under particular safety precautions, for example by the input of a password or a user identification or the like. Since the ventilation device in flow-controlled ventilation primarily attempts to administer the volume that is set, what may sometimes arise in this form of ventilation are high peak pressures. Therefore, such an embodiment is a particularly secure way of reliably preventing disadvantageous peak pressures. In particular, at least one maximum pressure is stored as an alarm limit.

In particular, the defined respiratory gas flow has a flow rate within the range of 0 to 90 l/min and preferably 1 to 80 l/min and particularly preferably 2 to 60 l/min. Other flow rates are also possible. In particular, the ventilation device is settable to such a flow rate. The defined respiratory gas flow can have a flow rate for infants, per selectable default setting, within the range of 1-10 l/min. In particular, it is possible to carrying out ventilation as intended with such a flow rate. In particular, a respiratory gas flow having a flow rate of 0 to 300 l/min is settable.

In all embodiments, the system is suitable and designed for being operated as intended with a nasal cannula as patient interface. In particular, an open nasal cannula is provided. In particular, the respiratory gas stream is administrable to the patient with a nasal cannula. In particular, at least one nasal cannula is connectable to the ventilation device. In particular, the nasal cannula is connectable to the hose device. It is also possible that the hose device is permanently and/or detachably connected to a nasal cannula. In particular, the ventilation hose is couplable to a nasal cannula. In particular, the system comprises at least one nasal cannula. Such a nasal cannula is particularly advantageously suitable for high-flow ventilation and provides the patient with a particularly high level of comfort. It is also possible that the system is operable with another suitable patient interface and, for example, with a tracheostomy connector.

In an advantageous further development, the system comprises a nasal cannula having a nozzle for each nostril, each nozzle being at least partly insertable into the respective nostril and each nozzle having a diameter dimensioned such that the respective nostril is not tightly sealed in the event of an inserted nozzle.

Each nozzle can, for example, have a diameter which is less than 9/10, preferably less than 8/10 and more than 6/10, of the diameter of the respective nostril.

The method according to the invention serves for operating a system as described above. In particular, the above-described systems are suitable and designed for being operating as per the method according to the invention.

The parameter is especially a pressure. The parameter monitored by the sensor device is especially the pressure at which the ventilation device must admit the respiratory gas stream in order to maintain the defined respiratory gas flow over the breathing activity of the patient and especially to keep said respiratory gas flow at a certain flow rate or desired flow rate and/or at at least one other characteristic variable that is characteristic of the respiratory gas flow.

The parameter describes especially the pressure of the respiratory gas stream in the ventilation device and/or at an outlet of the ventilation device and especially at an interface for connection to a hose device and/or a patient interface. The parameter describes especially a pressure adjustment necessary for bringing the actual value of the respiratory gas flow to a desired value that is required. It is also possible that the parameter is at least one other variable characteristic of a pressure. In this case, the parameter is, for example, a fan rotational speed or the like.

The sensor device comprises especially at least one sensor for capturing the parameter. The sensor device comprises especially at least one pressure sensor and/or at least one rotational speed sensor and/or at least one flow sensor and/or some other suitable sensor.

In particular, the control device is suitable and designed for ascertaining the pressure which must be applied by the ventilation device in order to keep the defined respiratory gas flow constant. In particular, the control device is suitable and designed for ascertaining the pressure which must be applied by the ventilation device in order to keep the respiratory gas flow influenced by breathing activity at a defined desired value. In particular, the control device is suitable and designed for regulating the defined respiratory gas flow to a certain flow rate by performing by means of the ventilation device a pressure adjustment and/or at least one adjustment of the rotational speed of a fan device. The pressure of the respiratory gas stream is set especially by a rotational speed of a fan device. It is also possible that the pressure is set by a specific opening and closing of a pressurized gas source. The control device is especially suitable and designed for regulating the defined respiratory gas flow by means of pressure adjustments. The control device can especially perform pressure adjustments in order to keep the defined respiratory gas flow at a certain flow rate and, at the same time, to preferably compensate for the breathing activity of the patient.

Using the system according to the invention, it is possible to carry out especially flow-controlled or high-flow ventilation. In particular, the ventilation device is controllable by means of the control device. In particular, the ventilation device is controllable by means of the control device for specifying a defined respiratory gas flow.

The defined respiratory gas flow is especially a constant flow. The defined respiratory gas flow is especially defined by a specified flow rate. The defined respiratory gas flow can also be defined by at least one other characteristic variable that is characteristic of a volume flow. In the context of the present invention, the flow rate is also referred to as flow. The respiratory gas flow can also be referred to as flow. The defined respiratory gas flow describes especially the quantity of the respiratory gas provided to the patient or of the respiratory gas flowing into the patient in relation to time. Flow-controlled and high-flow ventilation are used here as synonyms.

### BRIEF DESCRIPTION OF THE DRAWINGS

Further advantages and features of the present invention will become apparent from the description of the exemplary embodiments, which are elucidated below with reference to the accompanying drawings.

In the drawings:

FIG. 1 shows a purely schematic representation of a system according to the invention in a perspective view;

FIG. 2 shows a highly schematized chart relating to the functioning of the system; and

FIG. 3 shows a further highly schematized chart relating to the functioning of the system.

### DETAILED DESCRIPTION OF EXEMPLARY EMBODIMENTS OF THE INVENTION

The particulars shown herein are by way of example and for purposes of illustrative discussion of the embodiments of the present invention only and are presented in the cause of providing what is believed to be the most useful and readily understood description of the principles and conceptual aspects of the present invention. In this regard, no attempt is made to show details of the present invention in more detail than is necessary for the fundamental understanding of the present invention, the description in combination with the drawings making apparent to those of skill in the art how the several forms of the present invention may be embodied in practice.

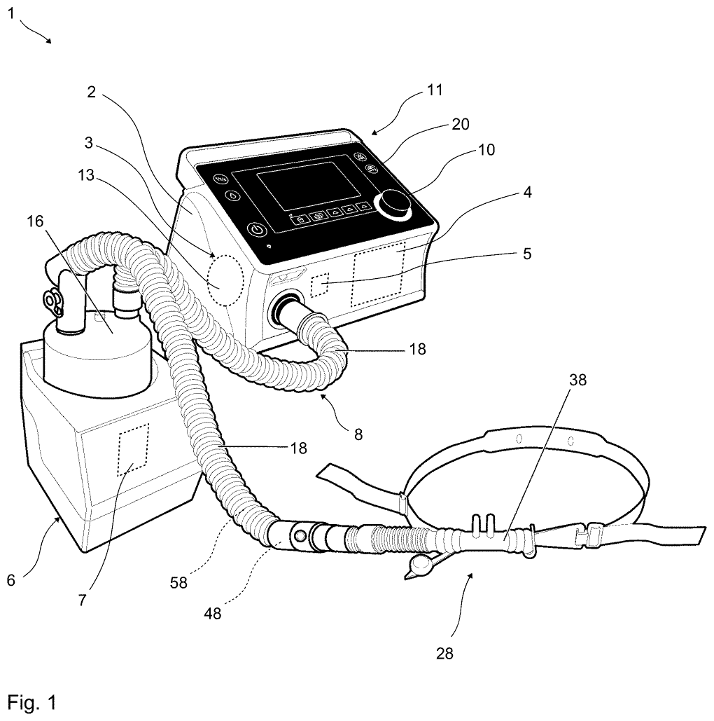

FIG. 1 shows a system 1 according to the invention for supplying respiratory gas or ventilation, comprising at least one ventilation device 2, which is provided here by a ventilator 11. The system 1 shown here is operated in accordance with the method according to the invention. The ventilation device 2 is equipped with a respiratory gas source 3 for generating a respiratory gas stream, which is provided here by a fan device 13 inside the housing.

By means of a fan, the fan device 13 generates the respiratory gas stream, which is supplied to the patient via a hose device 8 coupled to the ventilation device 2. For example, an electromotor with fan wheel is provided. In addition or as an alternative, a pressurized gas source can also be provided as a respiratory gas source 3.

Here, the ventilator 11 comprises an operating device 10 and a display device 20. In this connection, it is also possible to provide combinations of operating device 10 and display device 20, for example in the form of a touch-sensitive display area or touchscreen.

The system 1 is designed for a flow-controlled ventilation or high-flow ventilation. The ventilation device 2 is controlled by a control device or control unit 4 which is arranged inside the housing and is not visible here. For the flow-controlled ventilation, the control device 4 sets the ventilation device 2 to a defined respiratory gas flow. As a result, a defined respiratory volume is administered to the patient within a specified inspiration time. In one embodiment, the system 1 can also be usable for pressure-supported or pressure-controlled ventilation at a defined flow.

Here, the system 1 is equipped with a hose device 8 adapted or optimized for the flow-controlled ventilation. For example, a respiratory gas flow with a flow rate of 0 to 300 l/min is set. For this purpose, a ventilation hose 18 which is coupled with the respiratory gas source 3 and a patient interface 28 is provided.

The patient interface 28 is designed as an open nasal cannula 38. The nasal cannula 38 can also be referred to as nasal prongs. Advantageously, the hose device can also be used with a tracheostomy connector. Such a nasal cannula 38 is generally considerably more comfortable for the patient than, for example, a solid respiratory mask and a particular advantage of the flow-controlled ventilation. In this connection, the nasal cannula 38 is hung loosely in the nose and flushed with an appropriately high predefined respiratory gas flow (high-flow or else constant flow). As a result, carbon dioxide is washed out of the upper respiratory tract before inhalation and a small positive overpressure arises, meaning that especially the inhalation can be supported in a particularly comfortable manner. The nasal cannula allows a constant leakage, whereas the respiratory mask substantially avoids a leakage.

So that the mucous membranes are not dried out as a result of the constant air stream, the respiratory gas stream is specifically humidified and heated here. To this end, the system 1 is equipped here with a humidification device 6 and a heating device 7. The heating device 7 is integrated in the humidification device 6. The control device 4 additionally activates the humidification device 6 and/or the heating device 7 at least occasionally in order to humidify and/or heat the respiratory gas.

The ventilation hose 18 is two-part and a first part thereof extends from the ventilation device 2 to the humidification device 6. A second part of the ventilation hose 18 then extends from the humidification device 6 to the nasal cannula 38.

The humidification device 6 is equipped with a reservoir 16 for water. The water is vaporized and/or atomized and mixed with the respiratory air streaming through the ventilation hose 18. At the same time, the respiratory air streaming through the humidification device 6 is specifically heated via the heating device 7.

For an optimal setting of humidity and/or temperature of the respiratory gas stream, the hose device 8 is equipped here with a sensor unit 48. The output of the humidification device 6 and heating device 7 is then appropriately regulated via said sensor unit 48.

In addition or as an alternative to the heating device 7, the hose device 8 can be designed here as a heating hose. To this end, the ventilation hose 18 comprises its own heater 58. In this connection, the heater 58 is nondestructively separable from the ventilation hose 18, meaning that the heater 58 can be further used with a new ventilation hose 18 after hose exchange. The heater 58 can be regulatable via the sensor unit 48.

The system 1 shown here is equipped with a sensor device 5, which is accommodated here nonvisibly in the ventilator 11.

Via said sensor device 5, the control device 4 captures the pressure which must be applied by the ventilation device 2 for maintaining the defined respiratory gas flow in order to counterbalance the breathing activity of the patient. For example, what is measured for this purpose is the pressure at the instrument outlet, which pressure is required by the ventilation device 2 in order to keep the defined respiratory gas flow constant. Said pressure constantly changes. For example, to maintain the defined respiratory gas flow, less pressure is required during inhalation than during exhalation.

By contrast, more pressure is required during exhalation, since the patient is breathing against the administered respiratory gas flow. The sensor device 5 comprises, for example, at least one pressure sensor and/or at least one flow sensor.

The control device 4 registers the course of the captured pressure over time and saves said course as a pressure profile. By means of said pressure profile, the control device 4 can determine here a characteristic variable that is characteristic of the breathing activity of the patient. For example, the control device maps the breathing pattern via the pressure profile. From the breathing pattern, it is then possible to identify various breathing events and situations.

In turn, a response can be made thereto, for example with appropriate settings of the ventilation device and especially flow changes. Moreover, the breathing events or breathing patterns can be transmitted by telemonitoring. For example, the AHI can be ascertained therefrom.

The control unit 4 is configured and designed here for identifying breathing efforts from the pressure signal and/or from the flow signal from the pressure sensor device 5 and/or the flow sensor device 5. Moreover, the control unit 4 is configured and designed for identifying periodic breathing or apnea from the pressure signal and/or from the flow signal from the pressure sensor device 5 and/or the flow sensor device 5.

The system 1 is, for example, also designed and configured such that an admixing of oxygen, via an internal or external oxygen source not depicted in detail here, takes place up to 300 l/min or greater.

The system 1 is, for example, designed and configured such that the control unit 4, taking into account the signals from the flow and/or pressure sensor device 5, activates an oxygen source for admixing of oxygen, which is modulated in a breathing phase-dependent manner. For example, one intention is to control the admixing of oxygen such that more oxygen is available to the patient in phases of inspiration than for expiration.

The system 1 is, for example, designed and configured such that a pulse oximeter or a CO2 meter can be adapted, which then communicates with the system 1 and/or is supplied with energy thereby. Via the measurement values of these instruments, it is possible for the user—or an automatic algorithm—to set the correct flow rate and the correct admixing of oxygen. For example, the control unit takes into account measurement values from the pulse oximeter and/or the CO2 meter in the activation/control of the oxygen source and/or in the activation/control of the flow control module.

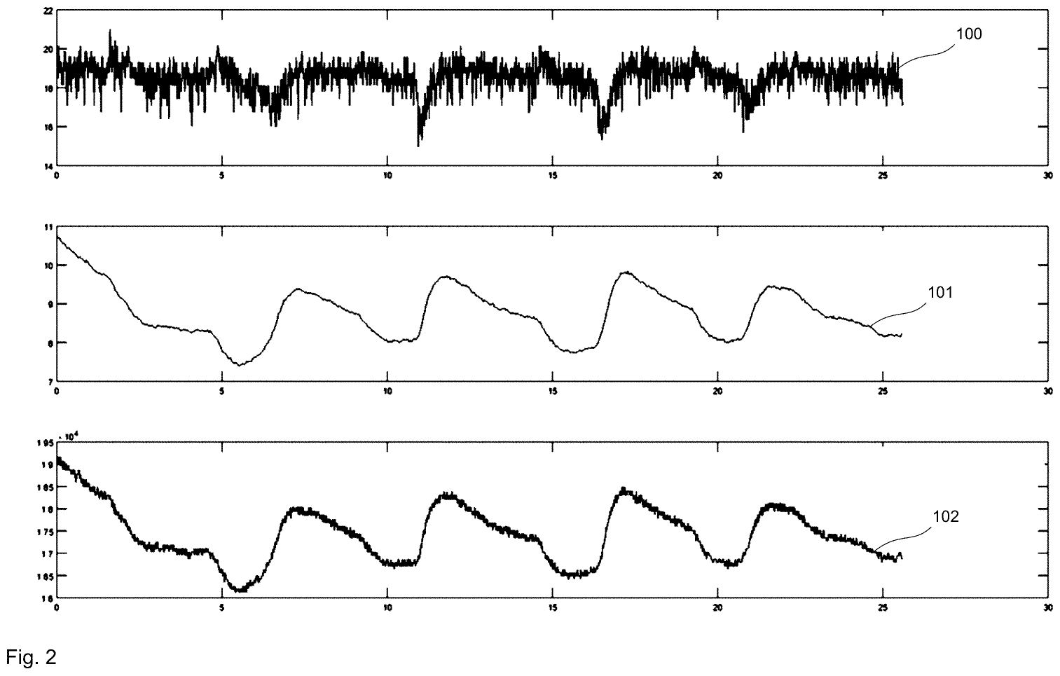

FIG. 2 shows a flow profile 100 and a pressure profile 101 and also a rotational speed profile 102 of the rotational speed of the fan device 13, which were recorded during an exemplary breathing activity or an exemplary operation of the system 1. In said figure, the top graph shows the flow profile 100, in which the flow rate in liters per minute was plotted against time. The middle graph shows the pressure profile 101, in which the pressure captured by the sensor device 5 was plotted over time. The bottom graph shows the rotational speed profile 102, in which the rotational speed in revolutions per minute of the fan device 13 was plotted against time. Here, the values were captured during an oral respiration of the patient.

Here, the flow that is set is 20 l per minute. It can be seen from the plot that the flow fluctuates within the range from 16 to 20 l/min. These fluctuations can be explained by the breathing activity of the patient. During inhalation, the flow increases. At the start of exhalation, it decreases. This can be explained by the back pressure which is caused by the patient when exhaling. During exhalation, the flow from the nasal cannula is slightly suppressed. At the start of inspiration, the respiratory gas can stream unimpeded from the patient interface.

It is apparent that the pressure fluctuates between 7.5 and 10 mbar. These fluctuations are caused by the breathing of the patient. With inhalation, the respiratory gas can stream more easily from the patient interface, and the pressure in the hose system decreases accordingly. With exhalation, the flow of the exhaled air opposes the flow of the respiratory gas. Since the control unit 4 attempts to keep the flow constant, it must increase the pressure during exhalation in order to allow the same flow output to stream from the patient interface.

It can be seen that the rotational speed fluctuates within a certain range. The fluctuations correlate with the fluctuations in the signal of the pressure profile 101. The fluctuations also correspond to the fluctuations in the signal of the flow profile 100. So that the flow is kept constant during inhalation, the control unit 4 must slightly lower the rotational speed. During exhalation, the control unit 4 attempts to keep the flow at a specified level. To this end, the rotational speed must be raised in order to increase the pressure and in order to ensure a constant flow via the increased pressure.

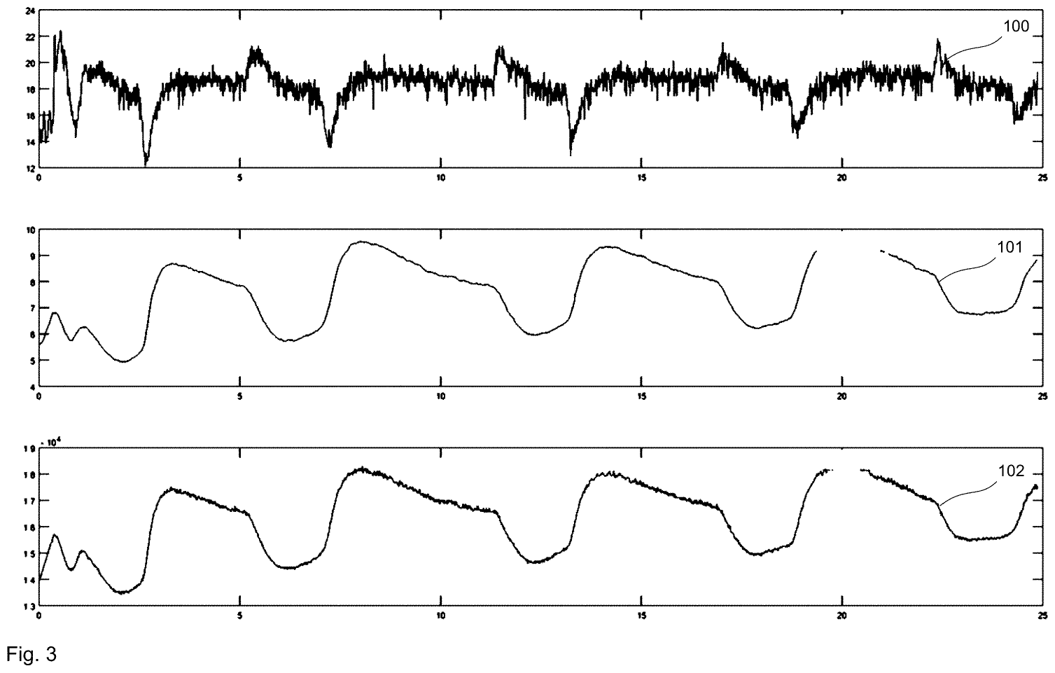

FIG. 3 shows, in analogy to FIG. 2, the flow profile 100 in the top graph and the pressure profile 101 in the middle graph and the rotational speed profile 102 in the bottom graph. In contrast to FIG. 2, the values were captured here during a nasal respiration. This involved the mouth being closed and the patient breathing through the nose.

What is used for the identification of the AHI as characteristic variable is, for example, a decrease in the required pressure fluctuation for correction of the respiratory gas flow by x %. The decrease in pressure fluctuation X is, in this case, preferably between 30% and 50% for hypopneas and/or between 70% and 100% for apneas. As an alternative or in addition, a length of time for the decrease in pressure fluctuation can be used; for example, the length of time is at least 5 seconds, preferably at least 10 seconds. The decrease in pressure fluctuation must therefore last at least 5 seconds in order to identify an apnea or hypopnea. The apneas and hypopneas per hour (AHI) can also be counted. In particular, at least one AHI value is saved per day. Moreover, a distinction is made between obstructive and central apneas and hypopneas. The correction of the constant respiratory gas flow via pressure can be effected approximately. In this connection, a preferred goal is that the respiratory gas flow fluctuates by less than 5 l/min and/or by less than 10%. If the correction is only successful outside a tolerance field, a weighted addition can alternatively be made to the pressure fluctuation and respiratory gas fluctuation to ascertain the breathing activity. In the event of identification of a lasting apnea and/or hypopnea, what can be provided is that the instrument identifies an incorrectly applied patient interface via this and then, for example, outputs an alarm.

For example, “Patient interface slipped” can be outputted. Furthermore, an identification of intermittent increased breathing as coughs can be provided. For example, the coughs per hour are counted and at least one value is saved per day.

In an advantageous embodiment, what is provided (especially as characteristic variable and/or by means of the characteristic variable) is an identification of decompensation. This is understood to mean especially an identification of the suspicion of an exacerbation. The identification is, for example, achieved via a rise in the respiratory rate, especially to over 20 bpm or by 20%, and/or via a drop in the basal oxygen saturation, especially to below 90% or 85% or by 5%, and/or via a rise in the pulse rate and/or via a rise in the number of coughs per hour or per day and/or via a rise in the number of apneas per hour or per day and/or via a rise or drop in the usage duration.

In a likewise advantageous embodiment, a monitoring interface is provided. This can be designed for monitoring in the hospital/care home and encompass, for example, Bluetooth, PDMS interface, LAN, WLAN. The interface can be designed for offline monitoring at home and encompass, for example, an internal instrument memory, storage medium, communication via Bluetooth with an app. The interface can also be designed for telemonitoring and be designed, for example, as an IoT interface and encompass or be suitable for, for example, GSM, NB-IoT, LTE-M, 2G, 4G, 5G. The interface can transmit signals, especially respiratory gas pressure and respiratory gas flow. The interface can transmit saved statistics and especially mean values and/or medians and/or percentiles and/or histograms, which preferably relate to respiratory rate, pressure fluctuations, respiratory gas flow fluctuations, SpO2 values, pulse rate values, FiO2 values or describe them. The interface can transmit saved events and especially number of apneas and hypopneas per hour per day or per day, number of oxygen desaturations per hour per day or per day, number of coughs per hour per day or per day. The interface can transmit alarms and encompass, for example, interface alarms, frequency alarms and/or decompensation alarms. The interface can transmit usage durations and/or usage times especially together with selected setting parameters. In particular, setting parameters and/or instrument parameters and/or ventilation parameters can be altered via the interface. Preferably, the instrument can be authenticated, preferably with its serial number and/or a security key, for example a certificate.

In the case of an identification of breathing phase as characteristic variable, what is effected by means of the characteristic variable is preferably at least one measurement of the respiratory rate especially via the formula: 60/duration of one breath (in s). In particular, what is effected depending on the breathing phase as a characteristic variable is controlling of the admixing of oxygen (inspiratory more than expiratory). In particular, what is effected depending on the breathing phase as a characteristic variable is controlling of the humidification and heating of respiratory gas (inspiratory more than expiratory). In particular, what is effected depending on the breathing phase as a characteristic variable is controlling of the respiratory gas flow: inspiratory more than expiratory (bilevel high-flow.

To sum up, the present invention provides:
1. A system for supplying respiratory gas, which system comprises (i) at least one ventilation device comprising at least one respiratory gas source for generating a respiratory gas stream and (ii) at least one control device which is configured for generating at least one defined respiratory gas flow for ventilation by the ventilation device and also is configured for monitoring at least one parameter characteristic of a pressure by at least one sensor device and characterizing that pressure at which the ventilation device admits the respiratory gas stream for maintaining the defined respiratory gas flow in order to counterbalance breathing activity of a patient, the control device also being configured for registering a course of the parameter over time as a pressure profile.
2. The system of item 1, wherein the control device further is configured for determining at least one characteristic variable that is characteristic of the breathing activity of the patient on the basis of the pressure profile.
3. The system of item 1 or item 2, wherein the control device further is configured for identifying an inhalation by the fact that a pressure required for maintaining the defined respiratory gas flow decreases over time.
4. The system of any one of the preceding items, wherein the control device further is configured for identifying an exhalation by the fact that a pressure required for maintaining the defined respiratory gas flow increases over time.
5. The system of any one of the preceding items, wherein the control device further is configured for determining at least one breathing event on the basis of the pressure profile.
6. The system of any one of the preceding items, wherein the control device further is configured for determining at least one breathing pattern on the basis of the pressure profile and for examining the at least one breathing pattern for at least one breathing event.
7. The system of any one of the preceding items, wherein the control device further is configured for outputting at least one user interaction, for example an alarm, depending on the pressure profile and/or is configured for transmitting the pressure profile, especially a breathing event and/or breathing pattern identified by means thereof, to a network device.
8. The system of any one of the preceding items, wherein the control device further is configured for setting at least one instrument parameter of the ventilation device depending on the pressure profile and/or is configured for setting at least one ventilation parameter by means of the ventilation device.
9. The system of item 8, wherein the instrument parameter and/or ventilation parameter, preferably a flow rate of the respiratory gas flow, is set depending on an identified breathing pattern and/or breathing event.
10. The system of any one of the preceding items, wherein the control device is configured for setting at least one flow rate of the respiratory gas flow depending on a saturation of the respiratory gas flow with oxygen by the ventilation device.
11. The system of any one of the preceding items, wherein the control device is configured for setting an admixing of oxygen into the respiratory gas stream depending on the pressure profile and especially for modulating the admixing of oxygen in a breathing phase-dependent manner.
12. The system of item 10 or item 11, wherein the admixing of oxygen and/or an oxygen content in the respiratory gas flow is decreased by a certain extent when a flow rate of the respiratory gas flow is increased.
13. The system of any one of items 10 to 12, wherein the admixing of oxygen only takes place during an inhalation and/or does not take place during an exhalation and/or wherein the admixing of oxygen is increased during an inhalation compared to an exhalation.
14. The system of any one of the preceding items, wherein the system further comprises at least one humidification device for humidification of the respiratory gas stream and/or at last one heating device for heating of the respiratory gas stream.
15. The system of item 14, wherein the control device is configured for setting a humidification and/or heating of the respiratory gas stream depending on the pressure profile and especially for regulating it/them to a target temperature and/or target humidity of the respiratory gas flow.
16. The system of any one of the preceding items, wherein the system comprises a humidification device for humidification of the respiratory gas stream and at least one heating device for heating of the respiratory gas stream, the heating device being configured for heating the respiratory gas stream at least during inspiration to 36-38° C. and the humidification device being configured for humidifying the respiratory gas stream with a relative humidity within a range of 90-100%, preferably 95-99%.
17. The system of any one of items 15 and 16, wherein a breathing phase-controlled humidification and/or heating is settable by the control device.
18. The system of any one of items 15-17, wherein the control device is configured for setting the humidification and/or heating depending on a flow rate of the respiratory gas flow.
19. The system of any one of items 15-18, wherein the control device is configured for setting the humidification and/or heating depending on at least one sensor-captured variable and wherein the variable is preferably taken from a group of variables comprising at least: ambient temperature, ambient humidity, temperature in the system, humidity in the system, hose temperature, temperature at a patient interface, humidity at the hose end and/or at a patient interface.
20. The system of any one of items 15-19, wherein the control device is configured for at least approximately compensating for flow errors caused by the humidification and/or heating and/or other deviations by the ventilation device.
21. The system of any one of items 15-20, wherein the control device is suitable and designed for reducing the flow rate of the respiratory gas flow that is intended for the ventilation until a measure of the humidity of the respiratory gas flow reaches a threshold and/or until the temperature of the respiratory gas flow reaches a threshold.
22. The system of any one of items 15-21, wherein at least one drying mode is performable in the follow-up to ventilation.
23. The system of any one of items 15-22, wherein the humidification device is configured for withdrawing the water intended for the humidification from at least one reservoir prior to heating and for heating it separately from the at least one reservoir.
24. The system of any one of the preceding items, wherein the system further comprises at least one hose device comprising at least one ventilation hose couplable to the ventilation device for supplying the respiratory gas stream to a patient interface.
25. The system of item 24, wherein the hose device comprises at least one heatable ventilation hose which comprises at least one part of a sensor unit for capturing at least one variable characteristic of humidity and/or temperature.
26. The system of item 24 or item 25, wherein the hose device comprises at least one ventilation hose which is gradually heatable over its length.
27. The system of any one of items 24-26, wherein the hose device comprises at least one heatable ventilation hose comprising at least one heater separable from the ventilation hose, the heater being equippable and further usable as intended with a new ventilation hose in the event of a hose exchange.
28. The system of any one of items 24-37, wherein the hose device comprises at least one ventilation hose composed of a water-channeling material and/or comprises at least one heat-insulated ventilation hose.
29. The system of any one of the preceding items, wherein the control device is configured for administering a defined respiratory volume within a specified inspiration time by the ventilation device through the defined respiratory gas flow.
30. The system of any one of the preceding items, wherein the control device is configured for taking into account at least one stored maximum pressure for the defined respiratory gas flow.
31. The system of any one of the preceding items, wherein the defined respiratory gas flow has a flow rate within the range of 0-110 l/min, preferably 1-80 l/min, particularly preferably 2-60 l/min.
32. The system of any one of the preceding items, wherein the defined respiratory gas flow has a flow rate for infants, per selectable default setting, within the range of 1-10 l/min.
33. The system of any one of the preceding items, wherein the system is configured for being operated as intended with a nasal cannula as patient interface, the nasal cannula having a nozzle for each nostril, each nozzle being at least partly insertable into a respective nostril and each nozzle having a diameter dimensioned such that the respective nostril is not tightly sealed in the event of an inserted nozzle.
34. The system of item 33, wherein each nozzle has a diameter which is less than 9/10, preferably less than 8/10 and more than 6/10, of the diameter of the respective nostril.
35. The system of any one of the preceding items, wherein the system is configured for being operated as intended with a respiratory mask as patient interface, the respiratory mask being configured for enclosing at least a nose of the patient and for substantially preventing a leakage stream, the control device being configured for operating the fan device such that it generates a substantially constant flow counteracting the breathing activity of the patient.
36. A method of providing a patient with respiratory gas, wherein the method comprises connecting the patient to the system of any one of the preceding items.

### LIST OF REFERENCE NUMERALS

1 System
2 Ventilation device
3 Respiratory gas source
4 Control device
5 Sensor device
6 Humidification device
7 Heating device
8 Hose device
10 Operating device
11 Ventilator
13 Fan device
16 Reservoir
18 Ventilation hose
20 Display device
28 Patient interface
38 Nasal cannula
48 Sensor unit
58 Heater
100 Flow profile
101 Pressure profile
102 Rotational speed profile

What is claimed is:

1. A system for supplying respiratory gas, wherein the system comprises (i) at least one ventilation device comprising at least one respiratory gas source for generating a respiratory gas stream and (ii) at least one control device which is configured for generating at least one defined respiratory gas flow for ventilation by the ventilation device and also is configured for monitoring at least one parameter characteristic of a pressure by at least one sensor device, the at least one parameter characterizing that pressure at which the ventilation device admits the respiratory gas stream for maintaining the defined respiratory gas flow in order to counterbalance breathing activity of a patient, and the control device being configured for registering a course of the parameter over time as a pressure profile.

2. The system of claim 1, wherein the control device further is configured for determining at least one characteristic variable that is characteristic of the breathing activity of the patient based on the pressure profile.

3. The system of claim 1, wherein the control device further is configured for identifying an inhalation by the fact that a pressure required for maintaining the defined respiratory gas flow decreases over time and/or is configured for identifying an exhalation by the fact that a pressure required for maintaining the defined respiratory gas flow increases over time.

4. The system of claim 1, wherein the control device further is configured for determining at least one breathing event on the basis of the pressure profile and/or is configured for determining at least one breathing pattern on the basis of the pressure profile and for examining the at least one breathing pattern for at least one breathing event.

5. The system of claim 1, wherein the control device further is configured for outputting at least one user interaction depending on the pressure profile and/or is configured for transmitting the pressure profile to a network device.

6. The system of claim 1, wherein the control device further is configured for setting at least one instrument parameter of the ventilation device depending on the pressure profile and/or is configured for setting at least one ventilation parameter by means of the ventilation device.

7. The system of claim 6, wherein the instrument parameter and/or ventilation parameter is set depending on an identified breathing pattern and/or breathing event.

8. The system of claim 1, wherein the control device further is configured for setting at least one flow rate of the respiratory gas flow depending on a saturation of the respiratory gas flow with oxygen by the ventilation device.

9. The system of claim 1, wherein the control device is configured for setting an admixing of oxygen into the respiratory gas stream depending on the pressure profile.

10. The system of claim 8, wherein an admixing of oxygen and/or an oxygen content in the respiratory gas flow is decreased when a flow rate of the respiratory gas flow is increased.

11. The system of claim 1, wherein the system further comprises at least one humidification device for humidification of the respiratory gas stream and/or at last one heating device for heating of the respiratory gas stream.

12. The system of claim 11, wherein the control device is configured for setting a humidification and/or heating of the respiratory gas stream depending on the pressure profile.

13. The system of claim 1, wherein the system comprises a humidification device for humidification of the respiratory gas stream and at least one heating device for heating of the respiratory gas stream, the heating device being configured for heating the respiratory gas stream at least during inspiration to 36-38° C. and the humidification device being configured for humidifying the respiratory gas stream with a relative humidity within a range of 90-100%.

14. The system of claim 13, wherein the control device is configured for setting the humidification and/or heating depending on at least one sensor-captured variable and/or is configured for at least approximately compensating for flow errors caused by the humidification and/or heating and/or other deviations by the ventilation device.

15. The system of claim 1, wherein the system further comprises at least one hose device comprising at least one ventilation hose couplable to the ventilation device for supplying the respiratory gas stream to a patient interface.

16. The system of claim 15, wherein the hose device comprises at least one heatable ventilation hose which comprises at least one part of a sensor unit for capturing at least one variable characteristic of humidity and/or temperature and/or comprises at least one heatable ventilation hose which comprises at least one part of a sensor unit for capturing at least one variable characteristic of humidity and/or temperature and/or comprises at least one ventilation hose which is gradually heatable over its length and/or comprises at least one heatable ventilation hose comprising at least one heater separable from the ventilation hose, the heater being equippable and further usable as intended with a new comprises at least one ventilation hose which is gradually heatable over its length.

17. The system of claim 1, wherein the control device is configured for administering a defined respiratory volume within a specified inspiration time by the ventilation device through the defined respiratory gas flow and/or is configured for taking into account at least one stored maximum pressure for a defined respiratory gas flow.

18. The system of claim 17, wherein the defined respiratory gas flow has a flow rate within a range of 0-110 l/min.

19. The system of claim 1, wherein the system is configured for being operated with a nasal cannula or a respiratory mask as patient interface.

20. A method of providing a patient with respiratory gas, wherein the method comprises connecting the patient to the system of claim 1.
